(19) United States
(12) Patent Application Publication
(10) Pub. No.: US 2023/0284962 A1
(43) Pub. Date: Sep. 14, 2023

(54) SYSTEMS AND METHODS FOR DIAGNOSING, ASSESSING, AND QUANTIFYING BRAIN TRAUMA

(71) Applicant: Weneuro Inc., Tel Aviv (IL)

(72) Inventors: Wayne Dam, Port Coquitlam (CA); Vladimir Zeev Roich, Tel Aviv (IL)

(21) Appl. No.: 18/119,145

(22) Filed: Mar. 8, 2023

Related U.S. Application Data
(60) Provisional application No. 63/318,261, filed on Mar. 9, 2022.

(51) Int. Cl. A61B 5/00; G16H 50/20; A61B 3/14; A61B 3/113; G16H 30/40; G06T 7/00

(52) U.S. Cl. CPC A61B 5/4064; G16H 50/20; A61B 3/145; A61B 3/113; G16H 30/40; G06T 7/0012; G06T 2207/30004; G06T 2207/10016

(57) ABSTRACT

Systems and methods are described for diagnosing, assessing, and quantifying brain injuries, traumas, or concussions. The systems and methods described herein are non-invasive and based on the detection, measurement, and analysis of involuntary micromotions and/or fixational eye movements with respect to an individual's eyes, pupils, and head. Determinations as to brain injury are based, in part, on divergences between measurements associated with the subject and measurements contained in one or more datasets. The described systems and methods are accessible to subjects regardless of geographic location or access to medical professionals, and are not reliant on the cooperation of the subject such that the systems and methods described here are still effective when the subject is non-responsive.

This non-provisional application claims benefit of priority to U.S. Provisional Patent Application No. 63/318,261, titled “SYSTEMS AND METHODS FOR DIAGNOSING, ASSESSING, AND QUANTIFYING BRAIN TRAUMA” and filed Mar. 9, 2022, which is incorporated herein in its entirety for all purposes.

### BACKGROUND

The present disclosure relates generally to the field of diagnosing, and assessing the extent of, brain injuries and/or trauma. In many cases, time is of the essence when diagnosing and assessing such traumas because delay in treatment or restorative care can lead to further damage to the affected individual's health. A failure to promptly alert the affected individual to the injury or trauma may also result in the individual engaging in activities (e.g., driving, sleeping, etc.) that may be dangerous to themselves and others given their current condition.

Today, clinical diagnosis and assessment of brain injuries requires the use of advanced clinical machinery and imaging (e.g., a computed tomography scan (“CT scan”) or magnetic resonance imaging (“MRI”)). While these tools and processes can be effective, they suffer from several drawbacks.

For example, it is necessary to transport the affected individual to a site at which the clinical machinery is available. In situations where the affected individual is alone, this may lead to the individual placing themselves in harm's way by, for example, driving a vehicle when it is otherwise unsafe to do so. It is also possible, depending on where and when the affected individual suffered their injury, that the appropriate clinical machinery is geographically remote or is otherwise inaccessible until business hours the following day. Such instances lead to an unnecessary and dangerous delay in the diagnosis of a trauma.

Typically, the presence of highly trained personnel, both for operating the clinical machinery and evaluating the machinery's output, is also required. Additionally, the output of such clinical machinery is often subjective such that a formal diagnosis is somewhat dependent upon the opinion and skill of a clinician.

Other known techniques rely on an affected individual's voluntary movements or responses to stimuli in the course of testing. Such techniques cannot be employed in the testing of an unresponsive subject. Such techniques can also be inaccurate due to the individual's failure to perform an action as anticipated by the diagnostic equipment and/or failure to perform the action at a time anticipated by the diagnostic equipment.

As a result, a need exists for new and improved tools for diagnosing and assessing brain injuries. In particular, a need exists for new and improved systems and methods that are accessible to affected individuals regardless of the individual's geographic location, proximity to specialized equipment and medical personnel, or a time of day or day of the week. A need further exists for testing equipment and diagnostic tools that provide more objective outputs than existing tools, are less reliant on the subjective opinions and skill of medical personnel, and are less reliant on voluntary movements of the subject such that an unresponsive subject can be tested.

### SUMMARY

Examples described herein include systems and methods for diagnosing, assessing, and quantifying brain injuries or traumas including, for example, the evaluation of craniocerebral injuries of various etiologies. In particular, the systems and methods described herein are non-invasive and based on the detection, measurement, and analysis of involuntary micromotions and/or fixational eye movements with respect to an individual's eyes, pupils, and head. These systems and methods are aimed at solving the problems and drawbacks associated with existing diagnostic processes and equipment (e.g., CT scans and MRI's) and mitigating the risk of delays between the occurrence of an injury and the diagnosis of the injury. These systems and methods are further aimed at improving upon diagnostic techniques that measure voluntary movements of subjects, including but not limited to voluntary movements of the subjects' eyes, head, or limbs.

In examples, systems and methods described here include recording, by a camera, one or more videos of at least a portion of a subject's face. Within the one or more videos, one or more biomarkers associated with the subject can be identified. In further examples, once identified, involuntary micromotions or fixational eye movements associated with the biomarkers are measured over the duration of a testing period (e.g. between 20 msec and 7000 msec). In some examples, these measurements are compared to measurements from a dataset in order to determine whether any divergences exist between the biomarker measurements and the dataset. Divergences, whether between a subject's left and right side or between the subject's measurements and those of a healthy population group, can indicate brain injury. Conversely, the lack of a divergence between the same data can indicate the absence of a brain injury. In a further aspect, the lack of a divergence between the subject's measurements and those of a population group having confirmed brain injury can be indicative of brain injury in the subject.

The examples summarized above can each be incorporated into a non-transitory, computer-readable medium having instructions that, when executed by a processor associated with a computing device, cause the processor to perform the stages described. Additionally, the example methods summarized above can each be implemented in a system including, for example, a memory storage and a computing device having a processor that executes instructions to carry out the stages described. As used herein, the terms “computing device,” “user device,” and “mobile device” are interchangeable and can encompass any type of computing device, such as laptops, tablets, smart phones, and personal computers.

Both the foregoing general description and the following detailed description are exemplary and explanatory only and are not restrictive of the examples, as claimed.

### BRIEF DESCRIPTION OF THE DRAWINGS

FIG. 1 is a flowchart of an example method for diagnosing and assessing a possible brain injury.

FIG. 2 is an illustration of an example system for diagnosing and assessing a possible brain injury.

FIG. 3 depicts an example graphical user interface for diagnosing and assessing a possible brain injury.

FIG. 4 depicts an example graphical user interface for diagnosing and assessing a possible brain injury.

### DESCRIPTION OF THE EXAMPLES

Reference will now be made in detail to the present examples, including examples illustrated in the accompanying drawings. Wherever possible, the same reference numbers will be used throughout the drawings to refer to the same or like parts.

The systems and methods described herein for diagnosing, assessing, and quantifying brain injuries can be easily accessible to affected individuals regardless of the individual's geographic location, proximity to specialized equipment or specialized personnel, or the time of day or day of the week. Such systems and methods can also provide objective and personalized results without assessing the voluntary movements of a subject during testing.

An affected individual can assess the presence and/or extent of a brain injury by themselves using the described systems and methods. A user can also assess the presence and/or extent of an injury to themselves or another person without needing special medical qualifications or training.

Brain trauma may have external causes (e.g., concussion) and/or internal causes (e.g., cerebral hemorrhage). Regardless of cause, the systems and methods described here can be used both for an initial diagnosis of such injuries and for subsequent evaluations of the course of the injury.

Under normal conditions the properties of the right and left sides of the body correlate with each other and the difference between them is within a narrow range. In the case of head trauma, the hemisphere of the brain (right or left) where the main trauma has occurred suffers more. As a result of trauma, the correlation/synchronization of right and left side properties of the body can be significantly reduced.

The systems and methods described here can collect and/or measure motion associated with a number of biomarkers of a subject and detect divergences in biomarker motion between the right and left side of the individual's body. As used herein, “biomarkers” can be involuntary micromovements of an individual's eyes or head, including but not limited to ocular drift and tremor (e.g., micro-tremors of the pupil), oscillation and/or change in pupil or iris size, microsaccades (as opposed to voluntary saccades), oscillation of regulation of blood vessels (e.g., facial blood vessels), and oscillation of muscle tone. In some embodiments, the measurements are directed to fast processes and short oscillations such that the measurement process can be completed in under 10 seconds. In further embodiments, the measurements process can be completed in approximately 7 seconds or less. In still further embodiments, the measurements process can be completed between 20 msec and 7000 msec. Detected movements, in such embodiments, do not rely on saccades or smooth pursuit with respect to eye movements, both of which result from voluntary movement of the subject and require a testing sequence that requires more time to measure.

In one aspect, measurements for both sides of the affected individual's body or face can be performed either simultaneously or separately. In the latter case, the measurement of the properties of one side can act as a calibration measurement to analyze the properties of the other side.

In another aspect, the measurements can be performed either in a passive form (e.g. video recording of a face) or in an active form (e.g., “live” video imaging). In the latter case, in some examples, the system can prompt the user to perform an action. In some examples, the system can prompt the user or affected individual to look at their hand in front of them (or another object in close proximity) or a distant object such as a building across the street. While some of these prompts may result in voluntary movements of the subject, the biomarkers of the subject that are measured during the test may still be involuntary micromotions and/or fixational eye movements. Any significant divergence between properties of the individual's right side and that of his or her left side can be a sign of a brain injury. The degree of divergence can also be assessed either by comparison with, for example: (1) a previous measurement for the subject taken prior to the suspected brain injury; (2) a previous measurement for the subject after the suspected brain injury but prior to the current test; or (3) by comparison of the measurements with population data from, for example, a database comprising data associated with healthy individuals, injured individuals, or a combination of the two.

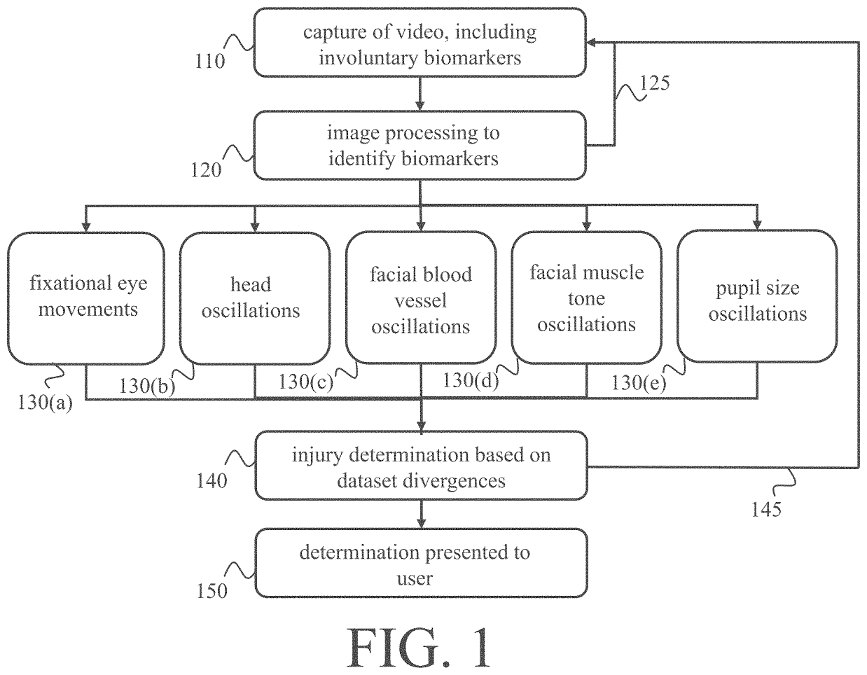

FIG. 1 depicts a process for diagnosing, assessing, and quantifying brain injuries based on an affected individual's involuntary micromotions and/or fixational eye movements with respect to an individual's eyes and/or head. In one aspect, at step 110, a computing device (described in more detail below) is used to record a video of an affected individual's face. The recording can be performed by a computing device (e.g., smartphone or laptop) with a suitable camera, processor, and memory. In some embodiments, the video includes the individual's head and face. In other embodiments, the video includes the individual's eyes and the skin or tissue around the eyes. In another aspect, a “live” image of the camera's view is presented to the user at a display of the computing device to help the user determine where and how to position the camera to adequately capture the user's eyes or head.

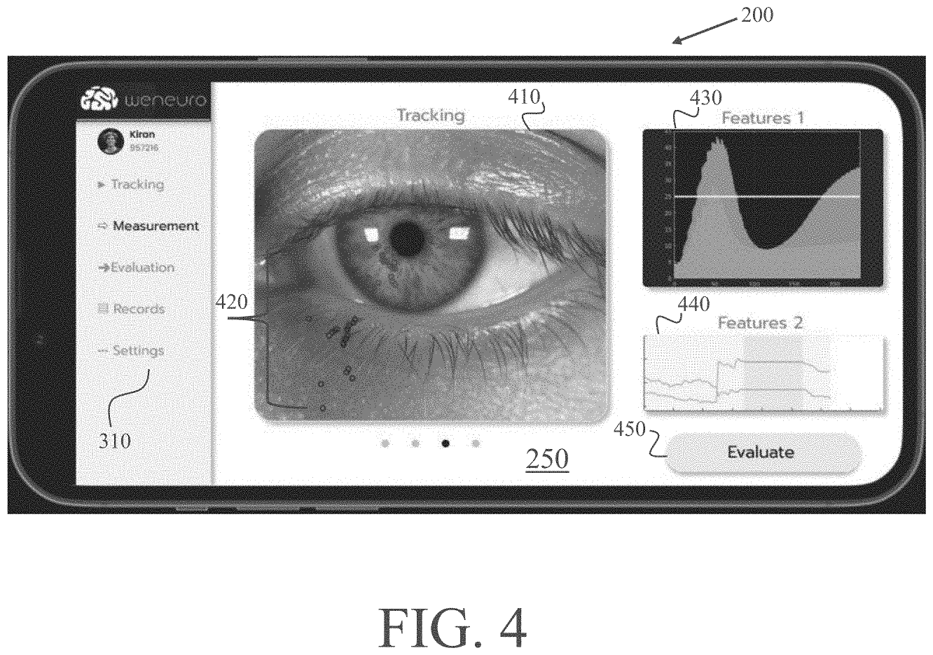

At step 120, the recorded image can be processed by the computing device. In one aspect, in order to minimize variables introduced by a subject's participation in the diagnosis, a set of involuntary biomarkers or image features are selected for measurement. The biomarkers contained in the recording can then be identified within the video (e.g., the biomarkers 420 identified in image 410 of FIG. 4) and tracked as time elapses (e.g., between 20 msec and 7000 msec). In some embodiments, the involuntary biomarkers can include, but are not limited to, fixational eye movements (e.g., microsaccades, drift, or tremor) and/or oscillations of the subject's head, facial blood vessels, facial muscle tone, or pupil size with respect to isolated locations identified in the subject's eye(s) (depicted, in one example, in FIG. 4, items 420).

At step 130, in some examples, the Kanade-Lucas-Tomasi (“KLT”) feature tracker can be used to track image features (e.g., biomarkers) within a recorded video. For motion magnification, in some embodiments, a two-dimensional dual-tree complex wavelet transform can be used. The data collected with respect to the selected biomarkers over the duration of a test period (e.g., between 20 msec and 7000 msec) can then be analyzed. In further examples, the collected data can include involuntary movements such as XY fixational eye movement, changes in pupil size, and oscillations associated with the subject's head, facial blood vessels, or facial muscle tone.

In still further examples, at step 130(a), any divergence in fixational eye movements (e.g., microsaccades, drift, and/or tremor) associated with the subject are assessed. In these examples, “divergence” is used to describe divergences: (1) between a subject's biomarkers on their left and right side (e.g., a divergence between fixational movements of the left eye and the right eye); (2) between a subject's biomarkers recorded in the present test vs. those recorded in a previous test; and/or (3) between a subject's biomarkers and the biomarkers of a population group contained in a database. In any case, divergences in the measured velocity and amplitude of fixational eye movements can be indicative of a brain injury or concussion. For example, a significant divergence (e.g., p<0.001) of microsaccades velocity and amplitude can be indicative of brain injury or concussion. In other examples, a divergence in eye drift and tremor can be indicative of neurodegenerative cerebellar disease and brain injury, respectively.

In another example, at step 130(b), head oscillation in the affected individual is also (or alternatively) recorded and tracked. In one aspect, accounting for head movement allows for the isolation of eye movement(s) because movement of the head can be accounted for and removed. In another aspect, head oscillation on its own can be an informative indicator of muscle tone (step 130(d)). Both increases and decreases in muscle tone can be indicative of brain injury or concussion, as can be a divergence in muscle tone between the individual's left and right side. For example, increases in muscle tone has been found in up to 13-20% of brain injuries and a loss in muscle tone has been found in up to 54% of concussion-related sports incidents.

In further aspects and similar to muscle tone, at step 130(c), oscillations in the subject's facial blood vessels can be recorded and tracked. In one aspect, small changes in skin color which can be detected by the camera are sufficient to detect flowing blood in facial blood vessels. Using video plethysmography, for example, involuntary micromotions associated with blood volume pulsations in facial tissue can be detected and measured over the testing time period. Changes or divergences in such micromotions can be indicative of brain injury or concussion.

In another example, at step 130(e), oscillations in pupil size of the affected individual's right and left eye over the course of the testing period can be analyzed. The size of an individual's pupils can change continuously, for example, in response to variations in ambient light levels. This process is modulated by cognitive brain function and any changes in brain function (e.g., changes resulting from an injury or concussion) can cause a change in how an individual's pupils respond to changes in ambient light. In some examples, significant divergences between an affected individual's maximum pupil diameter, papillary recovery time, constriction velocity, and latency of the pupillary light response can be detected and measured. Divergences exceeding a determined threshold can be indicative of brain injury or concussion. Ambient light and any changes thereto over the course of a test can also be measured by, for example, data received from a light source (e.g., a flash or flashlight of a smartphone) or data derived from the recording of the individual's face (e.g., brightness and/or changes to skin tone of the recorded individual). Any such changes in ambient light can then be accounted for when assessing oscillations in pupil size or other fixational eye movements.

In some embodiments, the unit of measurement for the movement of tracked image features (e.g., biomarkers 420 in FIG. 4) within a recorded video is degrees per time period (e.g., 5° in 2 seconds or, in another example, 3°/second). Eye movement can be measured as a rotation of the eyeball within the subject's head so rotation of the eyeball can be measured in degrees. In further embodiments, tracked image features (e.g., biomarkers 420 in FIG. 4) can be tracked as pixels identified and followed in an image/recording. In one example, if the distance between the camera and the eye is known, a pixel in the image/recording can be converted to, for example, millimeters (if XY movement over time is the unit of measurement) or degrees (if rotational movement over time is the unit of measurement).

As described here, any of steps 130(a)-(e) can be executed to the exclusion of any of the other steps. Similarly, any of steps 130(a)-(e) can be executed together, simultaneously, or in addition to any of the other steps.

At step 140, regardless of which biomarkers are identified, measured, and analyzed, the measured results associated with the subject's biomarkers can be compared to a dataset to determine the presence or absence of a brain injury or concussion. In one example, the subject's biomarker data can be compared to a database comprising historical data associated with the subject. For instance, the collected biomarker data can be compared to historical data associated with the subject at a time prior to the suspected brain injury and/or associated with a time when the subject was deemed healthy. Alternatively, in order to gauge the progression of an injury, the collected biomarker data can be compared to historical data associated with the subject at a time after the suspected brain injury but prior to the current test.

In other examples, the subject's biomarker data can be compared to a database comprising historical data associated with a population group. In some instances, the historical data can be associated with a healthy population group, a population group suffering from confirmed brain injuries or concussions, or some a combination of the two. A divergence between the measured biomarkers of the subject and those associated with healthy or uninjured individuals can be indicative of a brain injury or concussion. For example, a divergence in the subject's biomarker(s) outside a predetermined value such as one or two standard deviations from a healthy population group can be indicative of a brain injury. Conversely, a convergence (or lack of divergence) between the measured biomarkers of the subject and those associated with individuals suffering from a brain injury or concussion can also be indicative of a brain injury or concussion in the affected individual.

In another aspect, rather than (or in addition to) comparing the measured biomarkers to a historical dataset, an affected individual's biomarkers can be compared between their left and right side. Divergences between the left and right side of the individual can be indicative of a brain injury or concussion in the subject. For example, a divergence between the biomarker(s) of the left and right side outside a predetermined value such as one or two standard deviations can be indicative of a brain injury or concussion.

At step 150, the results of the analysis can be presented to the user via a user interface (“UI”) located at the computing device. In some examples, the presentation can include an indication of whether a brain injury or trauma is present and, if so, whether to seek medical attention. In further examples, the UI can include an indication of the severity of the injury and likely prognosis.

In further examples, the testing may not be able to proceed or the analysis may be inconclusive based, at least in part, on conditions present during the testing period (e.g., between 20 msec and 7000 msec). For example, ambient lighting during the testing period may have been inadequate to accurately detect, measure, or analyze one or more biomarkers. Alternatively, or additionally, the computing device may have detected physical movement of the camera (e.g., using an accelerometer present in the computing device) during the testing period that exceeds an acceptable threshold. In such cases where it is determined at step 120 that the testing conditions are inadequate, at step 125, instructions can be presented to the user via the UI to repeat the test under more favorable conditions. For example, the UI may instruct the user to move to a location with more ambient light or to perform the test again without moving the computing device during the test. In other examples, at step 125, the UI may instruct the user to repeat the test and prior to doing so, the computing device can automatically take remedial action(s) to resolve any problem(s) associated with the first test. For instance, in a situation where detected ambient light during the initial test is insufficient to ensure accurate test results, a flashlight or flash of the computing device may be instructed to activate and/or increase its intensity or brightness during a subsequent test.

In another aspect, if or when testing results at step 140 are inconclusive or additional imaging or data is needed, at step 145, instructions can be presented to the user via the UI to repeat all or a portion of a test and/or initiate a different testing protocol.

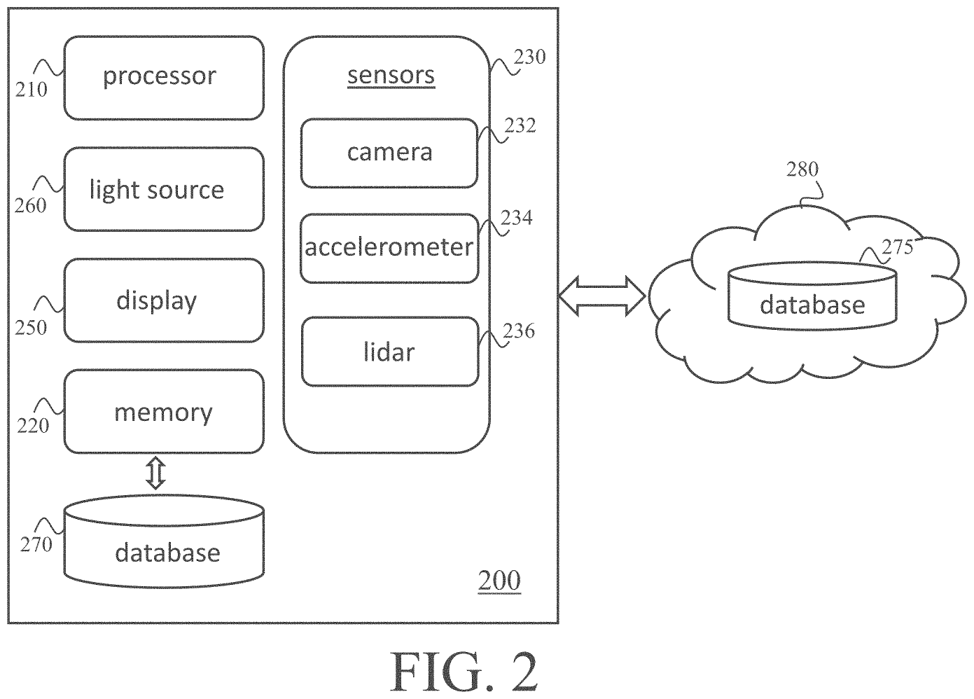

FIG. 2 depicts an example user device 200 for performing the processes described here. In some embodiments, the user device can comprise one or more of a processor 210, memory 220, sensors 230, and a display 250. The user device can be configured to measure the properties of a subject's right and left side, together and/or separately. Sensors 230 can include a camera 232 for recording video of a subject, an accelerometer 234 for detecting movement of camera 232/computing device 200, and a lidar unit 236 for measuring the distance between camera 232/computing device 200 and the subject. The approximate distance between camera 232/computing device 200 and the subject or the subject's face can be used to extract facial properties more accurately. In addition to the sensors, in some embodiments, the user device can further comprise a light source 260 such as a flash or flashlight.

In use, a user can hold computing device 200 in a way that the subject's face is visible to camera 232. In some embodiments, display 250 can depict instructions for the user that guide the user in positioning computing device for a test. For example, display 250 can instruct a user to move computing device 200 toward or away from the subject in order to adequately capture the appropriate biomarkers. In other examples, display 250 can instruct the user to move to an area with more or less ambient light. In still further examples, computing device 200 can instruct the user to steady the computing device in response to output from accelerometer 234 indicating the computing device is moving.

In another aspect, computing device 200 may automatically adjust in response to feedback from one or more sensors. For example, where computing device 200 detects too little ambient light in a recorded image from the camera, computing device 200 may instruct light source 260 to activate or increase in brightness. In another example, where accelerometer 234 indicates movement of computing device 200, the computing device's movements can be accounted for, and differentiated from, the subject's head and/or eye movements such that the subject's head and/or eye movements can be isolated. In this way, it is possible for head or eye movement to be recognized and measured even where computing device 200 is also moving.

Regardless of which biomarkers are identified, measured, and analyzed, computing device 200 may compare the measured biomarkers against historical data from a database 270. In some examples, the historical data corresponds with a population group comprising biomarkers for healthy individuals, injured individuals, or a combination of the two. In other examples, additionally or alternatively, database 270 can include historical data associated with the individual. For instance, the database can include historical data associated with the individual prior to suffering any injury. Alternatively, the database can include historical data associated with the individual after an injury but prior to the current test. In this manner, the systems and methods described here can determine whether a subject's injury is healing or progressing.

In another aspect, computing device 200 may be in communication with a network 280 such as the Internet. In such examples, some or all of the processing of biomarker information, including any comparisons between measured biomarkers and historical individual and/or population data, can be performed remotely (e.g., in the cloud). In other examples, some or all of the data within database 270 may be stored in remote database 275 in order to conserve memory space at computing device 200.

Whether some or all data is stored locally or in the cloud, and whether some or all of the processing of biomarker information occurs locally or in the cloud, in further examples, computing device 200 can further be configured to automatically alert a third party, such as a doctor or caregiver, when test results indicate a brain injury or concussion. Such an alert can include an indication of the subject's injury, a severity level of the injury, the user's location, and/or recommended treatment options for the user, for example.

In some embodiments, computing device 200 can receive input from sensors 230, including camera 232, evaluate measurements associated with one or more biomarkers in real-time, and provide real-time recommendations to the user via display 250 regarding the test results and/or how to perform or improve a subsequent measurement. For example, computing device can test left and right parts of the body independently (e.g., firstly, the right eye, secondly, the left eye). Computing device 200 can use the features of the images to adjust and/or compare two or more independent images (e.g., computing device 200 can use the sizes of the pupils or irises recorded for the left and right eyes to adjust the images to each other considering their sizes must be equal). In some embodiments, this can help to assess the distance between the camera and the user's face if auxiliary sensors such as lidar are not available or do not provide sufficient accuracy under the circumstances.

In some embodiments, computing device 200 can use any facial features to recognize head and/or hand movements during the image recording. For example, eyelashes can be used to recognize the pupil or iris movements against the head movements. The features can be the properties of the images in temporal and special domains. For example, color changes of the user's face within a period of time. In further or alternative embodiments, computing device 200 can also identify inconclusive tests, then computing device 200 can inform the user via display 150 and make suggestions to the user to perform additional measurements or tests.

In another aspect, display 250 can depict a UI used to guide a user through a test. In further examples, the UI can display step-by-step instructions for the user to carry out the test and, upon completion, display test results, a prognosis, and/or treatment options to the user. In the event of an inconclusive test or unfavorable testing conditions, the UI can also provide the user feedback for improving the test conditions and repeating the test.

The following examples describe tests that can be performed using computing device 200:

### Example 1

In some embodiments, the user may desire to make a self-assessment of his or her probable or possible brain trauma. Using the UI of computing device 200, the user can open an application configured to diagnose and assess brain injuries. The UI can suggest or prompt the user to direct camera 232 of computing device 200 at the user's face.

In such examples, a processing unit (“PU”) of computing device 200 can recognize in real-time the image of the user's face. The PU, together with camera 232, can record the image and perform preprocessing steps, including assessing the image quality and/or feasibility of biomarker extraction related to the left and right sides of the subject's face. In some examples, if the PU determines that biomarkers cannot be extracted from the current image with a required or threshold level of accuracy (e.g., the PU cannot detect a face in the image), in some embodiments, the PU can convey instructions, via the UI, to the user to bring computing device 200 closer to the user's face and/or place computing device 200 on a sturdy surface (e.g., a table or tripod) to reduce interference due to possible movement (e.g., a tremor of the user's hands). The instructions can be visual (e.g., icons, text, lights), audio (e.g., voice, beeps, or sounds), and/or tactile (e.g., vibrations). The PU, together with camera 232, can then record the image.

In further examples, the PU can then process the image to compensate for any movements of the user's head and/or hands (e.g., based on feedback from accelerometer 234). The PU can then perform biomarker extraction. In one example, the biomarker(s) can relate to the changes of color in the user's face within a period of time.

Regardless of the specific biomarker(s) measured, the PU can analyze the divergence between features of the left and the right sides of the user's face. Additionally, in some examples, the PU can compare or detect any divergence between the measured features and historical data derived from a population (e.g., a population segment of similar age, gender, prior health conditions, environment, etc.). In some examples, the population data can correspond to data indicative of brain trauma. In other examples, the population data can correspond to data indicative of no brain trauma. In further examples, the population data can correspond to data both indicative and not indicative of brain trauma;

Based on the foregoing comparisons and any detected divergences, in some examples, the PU can determine whether brain trauma is present in the user. In further examples, the PU can estimate a probability and severity of brain trauma for the user, optionally together with treatment options or instructions to seek further medical help. The PU can then inform the user of the results of the assessment via the UI.

### Example 2

In another example, a similar process including some or all of the steps set forth in Example 1 may be performed except instead of comparing the user's features against historical data for a population, the PU can compare the user's biomarkers with the user's own previously recorded biomarkers (e.g., the user's biomarkers recorded prior to any suspected trauma). Alternatively, the PU can compare the user's detected biomarkers to both historical data from a population and previously recorded features for the user. These two comparisons can be done simultaneously, sequentially (in either order), or in overlapping fashion.

### Example 3

In some embodiments, upon opening an application configured to diagnose and assess brain injuries, the UI can prompt the user to direct camera 232 of computing device 200 at the user's right eye, for example. The PU can recognize the image of the user's eye in real-time.

Similar to Example 1, the PU can assess the image quality and feasibility of feature extraction related to the user's eye movements. In some examples, if the PU can't extract features with the required accuracy or with a threshold accuracy (e.g., the PU cannot detect an eye on the image), the PU can convey instructions, via the UI, to the user to bring computing device 200 closer to the user's face and/or place computing device 200 on a sturdy surface (e.g., a table or tripod) to reduce interference due to possible movement (e.g., a tremor of the user's hands). The PU, together with camera 232, can then record and process the image.

The PU can then request, via the UI, that the user direct camera 232 at the user's left eye, for example. The PU can repeat the previously described steps with respect to the user's left eye. In this manner, independent images of each of the user's eyes are captured or recorded. The PU can then process the images to compensate for any movements of the user's head and/or hands, as described in previous examples. In some embodiments, for example, eyelashes can be used to recognize the user's pupil or iris movement versus the user's head movement;

The PU can then process the size of the user's left and right pupils or irises to determine or approximate the distance between the camera and each eye. The PU can then process the images to extract the power spectrum density (“PSD”) of each pupil's or iris's movements. The PU can detect any divergence between features of the user's left and right eye.

The PU can then compare any detected divergence between the features of the user's left and right eye with similar historical data derived from a population (e.g., a population segment of similar age, gender, prior health conditions, environment, etc.). In some examples, the population data can correspond to data indicative of brain trauma. In other examples, the population data can correspond to data indicative of no brain trauma. In further examples, the population data can correspond to data both indicative and not indicative of brain trauma.

Based on the foregoing comparisons and any detected divergences, in some examples, the PU can determine whether brain trauma is present in the user. In further examples, the PU can estimate a probability and severity of brain trauma for the user, optionally together with treatment options or instructions to seek further medical help. In other examples, the PU can determine or estimate the status of a prior brain trauma (e.g., whether the user's condition is improving, stable, or deteriorating). The PU can then inform the user about the assessment results via the UI.

### Example 4

In some embodiments, additionally or alternatively with respect to Example 3, the PU can compare the measured features with control data (e.g., either historical population data indicative of no brain trauma or previously recorded user data indicative of no brain trauma) to determine any divergence with respect to the measured features. For example, in some embodiments, the divergence of the features from the compared data set must exceed the normal divergence by more than some percentage (e.g., 60% or more) to indicate a high probability of brain trauma; however, if real divergence is 55% and signal-noise ratio of the measured PSD is low, then the PU may conclude that the measurements are not sufficiently accurate or that no brain trauma is present.

Like in previous examples, in another aspect, the PU can make a decision that an accurate determination cannot be made using the available data. In such examples, the PU can request, via the UI, that the user initiate additional measurements or repeat the performed measurements. Independent measurements may include the measurement of other features (e.g., features other than the user's eyes).

In some embodiments, the user can be asked to hold the SU with the accelerometer in their right hand outstretched forward. In such examples, some or all of the steps set forth above in Example 3 can be repeated, taking the movements detected by the accelerometer into consideration. The PU can then consider the features extracted from the measurements of the eyes, taking into account any hand movement, and make a determination regarding brain injury probability or the status of a prior brain injury for the user.

### Example 5

In another example, a similar process including some or all of the steps set forth in Examples 3 or 4 may be performed. The user can then be asked or prompted to look at a moving point of the UI screen instead of using their hands. In such embodiments, the alignment of the eye movements and the moving point can be analyzed.

### Example 6

In further embodiments, rather than the moving point on the UI screen described in Example 5 that can be used to track eye movement, the systems and methods described here can be used to measure gaze fixation, i.e., the absence of movement when the user's eyes should be still. In further embodiments, the user may be prompted to focus on a still, near (i.e., close by) object and then on a still, farther off object. In this manner, the systems and methods described here can detect the user's eyes' ability to fixate on objects. In alternative embodiments, the user may be prompted to focus their gaze on only one object, near or far off, rather than one then the other. Comparisons between the left and right eyes of the user, for example, can then be compared in a similar fashion to that described above with respect to other examples. Additionally or alternatively, any tremor present in either eye can be detected.

### Example 7

In another example, similar processes including some or all of the steps set forth in the previous examples may be performed. Additionally or alternatively, the user can be asked or prompted to look at a light (e.g., light source 260 or an external, independent light source). In a further embodiment, the user can be asked or prompted to look at a blinking light. In either case, the PU can record the user's pupil and/or iris reaction to the light (whether blinking or steady) in the user's left and/or right eyes and these images can be used as set forth above to detect and assess trauma.

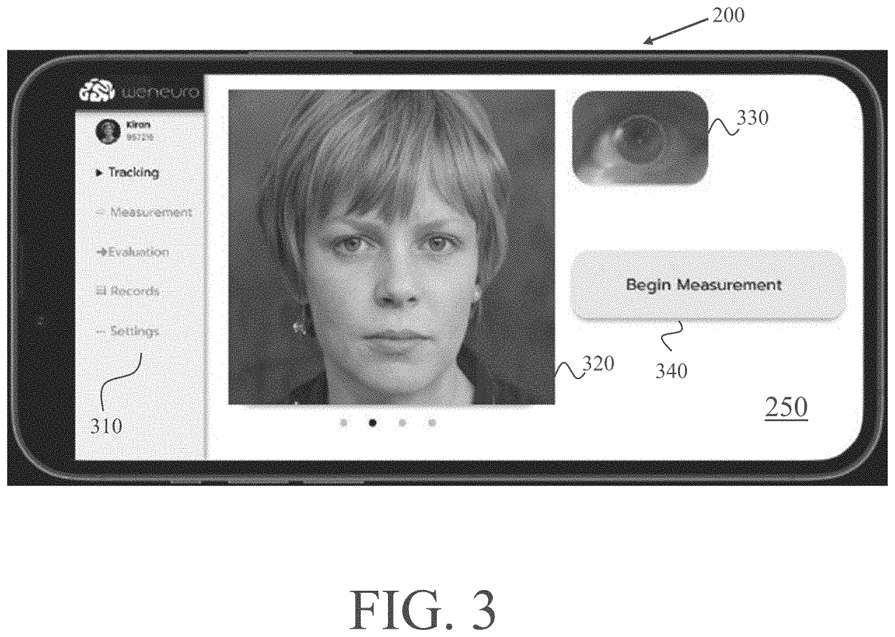

FIG. 3 depicts an illustrative example of UI/display 250 in use. In one aspect, the user is presented with progress bar 310. From progress bar 310, the user can track progress of a test as computing device 200 progresses from a “tracking” stage, to a “measurement” stage, to an “evaluation” stage. In one aspect, the tracking stage allows the user to confirm that computing device 200 has accurately located one or more areas of interest associated with the subject. For example, the UI in FIG. 3 depicts two images of the subject. A first image 320 depicts the user's entire face and a second image 330 depicts a zoomed in image of the subject's features relevant to testing. In the example shown, image 320 includes identifiers located on or around each of the subject's eyes. Likewise, image 330 depicts a zoomed-in image of one of the subject's eyes.

FIG. 3 further depicts an action button 340. If satisfied that computing device has accurately captured the relevant portions of the subject's face, the user can initiate the test by selecting action button 340.

FIG. 4 depicts another illustrative example of UI/display 250 in use. In one aspect, after the test is initiated, computing device 200 records and analyzes involuntary micromotions and/or fixational eye movements of the subject. In some examples, KLT feature tracking can be used to track image features (e.g., biomarkers) within a recorded video. For motion magnification, in some embodiments, a two-dimensional dual-tree complex wavelet transform can be used. The data collected with respect to the selected biomarkers over the duration of a test period (e.g., between 20 msec and 7000 msec) can then be analyzed.

In some examples, a live view of the video captured by the camera is displayed to the user at image 410. In further examples, within image 410, one or more biomarkers can be identified for tracking and analysis by, for example, KLT feature tracking. The UI can contain digital markings 420 superimposed on image 140 (e.g., depicted as small circles in image 140) to identify the selected biomarkers to the user.

In another aspect, the data acquired during the test (e.g., movement associated with one or more biomarkers) can be depicted and presented to the user in the form of graphs 430 and 440. Graphs 430 and 440 are only illustrative of examples of the type of measurements and visual representations that can be presented in the UI. For example, graph 430 can be a spectral power profile for one or both eyes in some embodiments. In another embodiment, graph 430 can depict pupil constriction, for instance, in degrees/time, XY-movement (horizontal and vertical)/time, and/or on a normalized scale of 0 to 1, for example.

In another example, graph 440 can depict movement of one or more biomarkers identified in a subject's eye(s) over time in some embodiments. In such embodiments, the x-axis can represent time and the y-axis can represent eye movement in degrees and/or XY-movement of the eye. In some embodiments, graph 400 can depict gaze direction, for example, in degrees/time and/or XY-movement (horizontal and vertical)/time.

In further examples, and similar to FIG. 3, the interface depicted in FIG. 4 can also include an action button 450. Upon completion of the testing cycle, the user can initiate the evaluation stage by selecting action button 450. In response to such a selection, in some embodiments, computing device will display to the user, via the UI, the results of the test.

Other embodiments of the systems and methods described here will be apparent to those skilled in the art from consideration of this specification and practice of the systems and methods disclosed herein. It is intended that the specification and examples be considered as illustrative only.

Though some of the described methods have been presented as a series of steps, it should be appreciated that one or more steps can occur simultaneously, in an overlapping fashion, or in a different order. The order of steps presented is only illustrative of the possibilities and those steps can be executed or performed in any suitable fashion. Moreover, the various features of the examples described here are not mutually exclusive. Rather any feature of any example described here can be incorporated into any other suitable example.

As used herein, the terms “injury,” “trauma,” and “concussion,” are used interchangeably. Likewise, the terms “subject” and “affected individual” are used interchangeably. The term “user” is intended to identify an individual operating computing device 200. The user may also be the subject or affected individual, or the user can use computing device 200 to diagnose and assess the brain injury of another individual who is the subject or affected individual.

Further, while computing device 200 and its components can be contained or integrated into in a single device, in other embodiments, computing device 200 can be comprised of multiple discrete devices, each containing one or more components of computing device 200 and each such discrete device being in communication with the other(s). For example, and without limiting other possibilities, one or more sensors 230 of computing device 200 (e.g., camera 232, accelerometer 234, and/or lidar 236) can be contained in a first discrete computing device and the remaining components (e.g., other sensors 230, processor 210, memory 220, display 250, light source 260, and/or database 270) can be contained in a second discrete computing device or distributed across multiple other discrete computing devices. Additionally, as discussed above, one or more components of computing device 200 (e.g., database 270) can be located in the cloud.

It is intended that the specification and examples be considered as exemplary only, with a true scope and spirit of the disclosure being indicated by the following claims.

What is claimed is:

1. A method for diagnosing and assessing brain injury in a subject, comprising:
recording, by a camera, one or more videos of at least a portion of a subject's face;
identifying one or more biomarkers associated with the subject within each of the one or more videos;
measuring involuntary micromotions or fixational eye movements associated with the identified one or more biomarkers within each of the one or more videos;
determining that a divergence exists with respect to a measurement associated with at least one of the one or more videos and a measurement associated with a dataset; and
based, at least in part, on the divergence, providing a notification indicating that a brain injury is likely.

2. The method of claim 1, wherein the one or more videos of at least a portion of the subject's face comprises at least a first video of at least a portion of one side of the subject's face and at least a second video of at least a portion of the opposite side of the subject's face.

3. The method of claim 2, wherein the measurement associated with the at least one of the one or more videos is a measurement derived from the first video, and the measurement associated with the dataset is a measurement derived from the second video.

4. The method of claim 1, wherein determining that the divergence exists includes determining that a difference between the measurement associated with the at least one of the one or more videos and the measurement associated with the dataset exceeds a predetermined threshold.

5. The method of claim 1, wherein the dataset comprises one or more biomarker measurements associated with a population group.

6. The method of claim 5, wherein the dataset comprises historical measurements collected from the subject.

7. The method of claim 1, wherein measuring the involuntary micromotions or fixational eye movements includes determining an amount of motion associated with the identified one or more biomarkers that is attributable to motion of the subject's head and taking the subject's head motion into account in determining the involuntary micromotions or fixational eye movements of the subject.

8. A computing system for diagnosing and assessing brain injury in a subject, comprising:
a processor;
a memory; and
a camera,
wherein the processor performs stages including:
recording, by the camera, one or more videos of at least a portion of a subject's face;
identifying one or more biomarkers associated with the subject within each of the one or more videos;
measuring involuntary micromotions or fixational eye movements associated with the identified one or more biomarkers within each of the one or more videos;
determining that a divergence exists with respect to a measurement associated with at least one of the one or more videos and a measurement associated with a dataset; and
based, at least in part, on the divergence, providing a notification indicating that a brain injury is likely.

9. The computing device of claim 8, wherein the one or more videos of at least a portion of the subject's face comprises at least a first video of at least a portion of one side of the subject's face and at least a second video of at least a portion of the opposite side of the subject's face.

10. The computing device of claim 9, wherein the measurement associated with the at least one of the one or more videos is a measurement derived from the first video, and the measurement associated with the dataset is a measurement derived from the second video.

11. The computing device of claim 8, wherein determining that the divergence exists includes determining that a difference between the measurement associated with the at least one of the one or more videos and the measurement associated with the dataset exceeds a predetermined threshold.

12. The computing device of claim 8, wherein the dataset comprises one or more biomarker measurements associated with a population group.

13. The computing device of claim 8, wherein the dataset comprises historical measurements collected from the subject.

14. The computing device of claim 8, wherein measuring the involuntary micromotions or fixational eye movements includes determining an amount of motion associated with the identified one or more biomarkers that is attributable to motion of the subject's head and taking the subject's head motion into account in determining the involuntary micromotions or fixational eye movements of the subject.

15. A non-transitory, computer-readable medium comprising instructions that, when executed by a processor of a computing device, cause the processor to perform stages for diagnosing and assessing brain injury in a subject, the stages comprising:
recording, by a camera, one or more videos of at least a portion of a subject's face;
identifying one or more biomarkers associated with the subject within each of the one or more videos;
measuring involuntary micromotions or fixational eye movements associated with the identified one or more biomarkers within each of the one or more videos;
determining that a divergence exists with respect to a measurement associated with at least one of the one or more videos and a measurement associated with a dataset; and
based, at least in part, on the divergence, providing a notification indicating that a brain injury is likely.

16. The non-transitory, computer-readable medium of claim 15, wherein the one or more videos of at least a portion of the subject's face comprises at least a first video of at least a portion of one side of the subject's face and at least a second video of at least a portion of the opposite side of the subject's face.

17. The non-transitory, computer-readable medium of claim 16, wherein the measurement associated with the at least one of the one or more videos is a measurement derived from the first video, and the measurement associated with the dataset is a measurement derived from the second video.

18. The non-transitory, computer-readable medium of claim 15, wherein determining that the divergence exists includes determining that a difference between the measurement associated with the at least one of the one or more videos and the measurement associated with the dataset exceeds a predetermined threshold.

19. The non-transitory, computer-readable medium of claim 15, wherein the dataset comprises one or more biomarker measurements associated with a population group.

20. The non-transitory, computer-readable medium of claim 15, wherein the dataset comprises historical data associated with the subject and collected prior to the brain injury.
